(19) United States
(12) Patent Application Publication
(10) Pub. No.: US 2017/0161892 A1
(43) Pub. Date: Jun. 8, 2017

(54) A METHOD FOR ACQUIRING AND PROCESSING IMAGES OF AN OCULAR FUNDUS BY MEANS OF A PORTABLE ELECTRONIC DEVICE

(71) Applicant: SI14 S.P.A., Padova (IT)

(72) Inventors: Andrea TELLATIN, Padova (IT); Tommaso GUSEO, Padova (IT)

(73) Assignee: SI14 S.P.A., Padova (IT)

(21) Appl. No.: 15/323,427

(22) PCT Filed: Jul. 2, 2015
(86) PCT No.: PCT/IB2015/054990

§ 371 Date: Jan. 2, 2017

(30) Foreign Application Priority Data
Jul. 2, 2014 (IT) PD2014A000173

(51) Int. Cl. G06T 7/00; A61B 3/12; A61B 3/00; G06K 9/00; A61B 3/14

(52) U.S. Cl. CPC G06T 7/0012; G06K 9/00604; A61B 3/14; A61B 3/0025; A61B 3/12; G06T 2207/30041; G06T 2207/10016

(57) ABSTRACT

A method for acquiring and processing images of the ocular fundus by means of a portable electronic device (1), wherein the portable electronic device comprises a photographic camera or video camera (2) capable of acquiring a plurality of images at a predetermined frequency, and a processor capable of processing these images, the method comprising:
acquiring a plurality of images of the ocular fundus by means of the photographic camera or video camera of the electronic device,
mapping certain images selected from the plurality of acquired images, wherein the selected images are positioned relative to one another so as to form a map of the ocular fundus formed by the assembly of the selected images, and the positioning of each image relative to the other, previously selected, images is carried out within a maximum time interval of 125 milliseconds, and
joining the selected images or images corresponding to them according to the mutual positioning defined by the mapping, so as to generate a single image of the ocular fundus.

### TECHNICAL FIELD

The present invention relates to a method for acquiring and processing images of an ocular fundus (fundus oculi) of a human being, or more generally of an animal, the method being performed by means of a portable electronic device.

### BACKGROUND ART

The analysis of the ocular fundus is a widely used method in the diagnosis of various pathological conditions, including those directly related to the eye, for example retinopathies and maculopathies, and those of a general nature, for example diabetes and hypertension. As a rule, this type of analysis is performed by a specialist doctor, by means of an appropriate apparatus called an ophthalmoscope, which can be used to display the ocular fundus, thus enabling the doctor to develop a diagnosis. In purely general terms, an ophthalmoscope comprises a light source and an optical unit formed by a series of lenses which focus the light through the pupil and return an image of the inside of the eye.

Accessories have recently been designed and produced for converting some commonly used portable electronic devices, such as mobile telephones, smartphones or tablets, into ophthalmoscopes, all these devices currently being provided with image acquisition devices, such as video-photocameras, which may be of high quality.

Accessories of this type, described, for example, in international patent application WO 2004/017825 or in Italian patent application BS2013A000169, may comprise a light source and an optical unit positioned in such a way that, when the accessory has been assembled on to the smartphone, the optical unit is aligned with the lens of the video-photocamera integrated into the smartphone, so as to enable the latter to acquire images relating to the ocular fundus.

The use of these instruments substantially increases the possibility of performing analyses of the ocular fundus (and corresponding diagnoses), even in environments lacking in technical resources and/or specialized operators, for example in poor or disadvantaged parts of the world.

In fact, because of the widespread distribution of these portable electronic devices among the population, and the capacity of these devices to save acquired images and transmit them, over the Internet for example, it is possible for images of the ocular fundus to be acquired even by a non-specialist operator and then to be transmitted remotely to a doctor capable of analysing them and making a diagnosis.

The use of portable electronic devices such as smartphones or tablets also makes it possible for purely hospital-based examinations, conducted with bulky and expensive machinery, to be extended to peripheral and domestic situations, owing to the possibility of storing the acquired images and processing some of the data directly in the device responsible for the acquisition.

However, the potential of these new instruments is still restricted by a number of disadvantages.

A first disadvantage is the fact that the viewing angle of the video-photocameras and optical units of these portable devices is generally rather limited by comparison with professional medical devices for capturing images of the ocular fundus. Indeed, the framed portion of these video-photocameras is only a third of the framed portion of the professional devices. This makes it necessary to acquire a plurality of images for the complete exploration of the whole ocular fundus.

Additionally, a non-expert operator may find it difficult to know whether all the areas of the ocular fundus have been adequately covered by the images acquired by the video-photocamera.

It is therefore possible that the operator may acquire and remotely transmit one or more images which do not reveal the whole ocular fundus, thus preventing the doctor from making an adequate analysis. On the other hand, for fear of making this mistake, the operator may be induced to acquire a large number of images, even though these have many areas in common, which are therefore unnecessarily repeated. This may not only complicate the subsequent analysis by the doctor, who has to manage and analyse a large number of substantially equivalent photographs, but also give rise to considerable problems in the transmission of the images over the Internet.

Indeed, given that the image must have a high degree of resolution to be correctly examined, a large number of images may excessively restrict or even compromise their correct remote transmission, owing to the large amount of data (in terms of MB) to be transmitted.

Another disadvantage of the known methods is that a new examination has to be conducted at a later time if the operator has not acquired all the portions of the retina necessary for the diagnosis (for example owing to the small size of the framed visual field, or the inadequate quality of the images acquired previously).

This inevitably leads to a deterioration of the examination conditions (dilation, luminous intensity, movement artefacts) and possibly the clinical conditions (appearance/disappearance of microbleeds or microaneurysms, exudates, angiosclerosis or other lesions).

Additionally, if this acquisition is carried out by a non-expert, the problem increases, since such a person will find it difficult to determine whether all the areas of the ocular fundus have been adequately covered by the images acquired by the video-photocamera.

Another disadvantage lies in the relative mobility between the patient and the portable acquisition device. Indeed, in most professional medical systems for analysis by means of images of the ocular fundus, the patient's head and the apparatus used for the examination must be limited in their movements unless the latter are decided on by the examining doctor.

However, one of the main difficulties in the acquisition of images of the ocular fundus by portable devices lies in the fact that the patient's head, and particularly his eye, is in continuous, unpredictable movement relative to the image capture apparatus, and therefore shows no deterministic correspondence. It will be readily appreciated that this situation results in a diversity in geometric terms (position, rotation) and chromatic terms (colours, reflections) among the plurality of acquired images, invalidating any direct comparison or joining (stitching) of the different images, since the final result would not be the entirety of the ocular fundus, but a series of images uncorrelated with one another. It is also known that the conventional algorithms used in the image stitching process suffer from limitations, mainly due to the distance between the object of the scene and the image capture point (see, for example, Satya Prakash Mallick, “Feature Based Image Mosaicing”, or Tuomas Makela, “Dental X-ray image stitching algorithm”, or Richard Szeliski (2004) “Image Alignment and Stitching”).

### DESCRIPTION OF THE INVENTION

The problem on which the present invention is based is that of providing a method for acquiring and processing images of an ocular fundus which is functionally designed to overcome at least some of the limitations described above with reference to the cited prior art.

This problem is resolved by the present invention by means of a method executed in accordance with the claims below.

In a first aspect, the present invention proposes a method for acquiring and processing images of the ocular fundus by means of a portable electronic device having a photographic camera or video camera capable of acquiring a plurality of images, and a processor capable of processing these images, in which method the following steps are provided:
acquiring a plurality of images of the ocular fundus by means of the video-photocamera of the portable electronic device,
mapping certain images selected from the plurality of acquired images, wherein the selected images are positioned relative to one another so as to form a map of the ocular fundus formed by the assembly of the selected images, and wherein the positioning of each image relative to the other, previously selected, images is carried out within a maximum time interval of 125 milliseconds, and
joining the selected images or images corresponding to them according to the mutual positioning defined by the mapping, so as to generate a single image of the ocular fundus.

In this way, all the data required for the graphic display of the ocular fundus are assembled in a single image which, by eliminating the problem of the repetition of common areas between different images, becomes much less cumbersome overall than the sum of a plurality of separate images, and is therefore easier to transmit over the Internet.

Furthermore, the provision of a single image representing the whole ocular fundus considerably facilitates the doctor's analytical work, since he no longer has to search for areas of interest among a large number of separate images, consequently minimizing the effects of local variations that are inevitably present when a plurality of discrete images are acquired, as these may create artefacts indicative of pathologies that are not actually present.

Another important advantage is due to the fact that, with this method, the mapping step can be carried out in real time relative to the acquisition of the images.

In a second aspect, the invention proposes a software application, loadable into a portable electronic device comprising a photographic camera or a video camera capable of acquiring a plurality of images and a processor capable of processing the images, wherein the software application is capable of controlling the acquisition and processing of images of the ocular fundus by means of the portable electronic device using the method according to the previous aspect.

In a third aspect, the invention proposes a portable electronic device, comprising a photographic camera or a video camera capable of acquiring a plurality of images and a processor capable of processing these images, into which is loaded a software application according to the preceding claim.

### BRIEF DESCRIPTION OF THE DRAWINGS

The characteristics and advantages of the invention will be more fully apparent from the detailed description of a preferred embodiment thereof, illustrated, for guidance and in a non-limiting way, with reference to the attached drawings, in which:

FIG. 1 is a schematic view of a portable electronic device designed to acquire and process images of an ocular fundus according to the method of the present invention;

FIGS. 2a and 2b show respective block diagrams of a first part of the method for acquiring and processing images of an ocular fundus by means of the device of FIG. 1, provided in accordance to the present invention,

FIG. 3 is a block diagram of a second part of the method provided in accordance to the present invention.

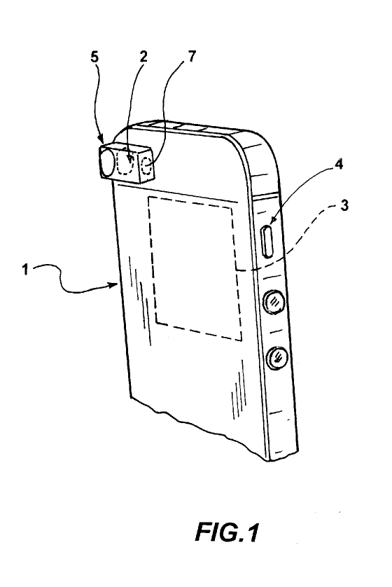

### PREFERRED EMBODIMENT OF THE INVENTION

In the drawings, the number 1 indicates the whole of a portable electronic device designed to operate according to the method of the present invention.

In the preferred embodiment described herein, the portable electronic device 1 is preferably a smartphone, but it may similarly take the form of a mobile telephone or a tablet.

By way of example, the portable electronic device 1 may be an iPhone® 5 smartphone produced by Apple Inc.

The portable electronic device 1 comprises an element for acquiring images, a processor capable of controlling both the acquisition and the processing of these images, and a memory 6 suitable for storing them. In particular, the image acquisition element is formed by a video-photocamera 2, capable of acquiring a plurality of images either in the form of single images or in the form of a video recording.

Preferably, the video-photocamera 2 is capable of acquiring images in high definition, according to the 1080p standard.

Preferably, also, the images are acquired by the video-photocamera 2 at a frequency of 30 frames per second (30 fps).

Additionally, the portable electronic device 1 preferably comprises a display 3, which can display a graphic interface capable of showing information to the user and receiving commands from him, together with means of a known type for connection to the telephone network and to the Internet.

The portable electronic device 1 is also provided with an accessory 5, coupled removably to the outer casing 4 of the device 1, which is suitably designed to allow accurate viewing of the ocular fundus by means of the portable electronic device 1. For this purpose, the accessory 5 comprises an optical unit positioned so as to enlarge the images entering the video-photocamera 2, and may, optionally, be provided with a light source capable of illuminating the visual field recorded by the video-photocamera 2.

Alternatively, the accessory 5 may be designed to make use of any light source 7 that may be present on the electronic device 1 and to guide it suitably towards the target to be recorded.

Exemplary embodiments of accessories 5 suitable for operation according to the method of the present invention are described in international patent application WO 2004/017825 and in Italian patent application BS2013A000169.

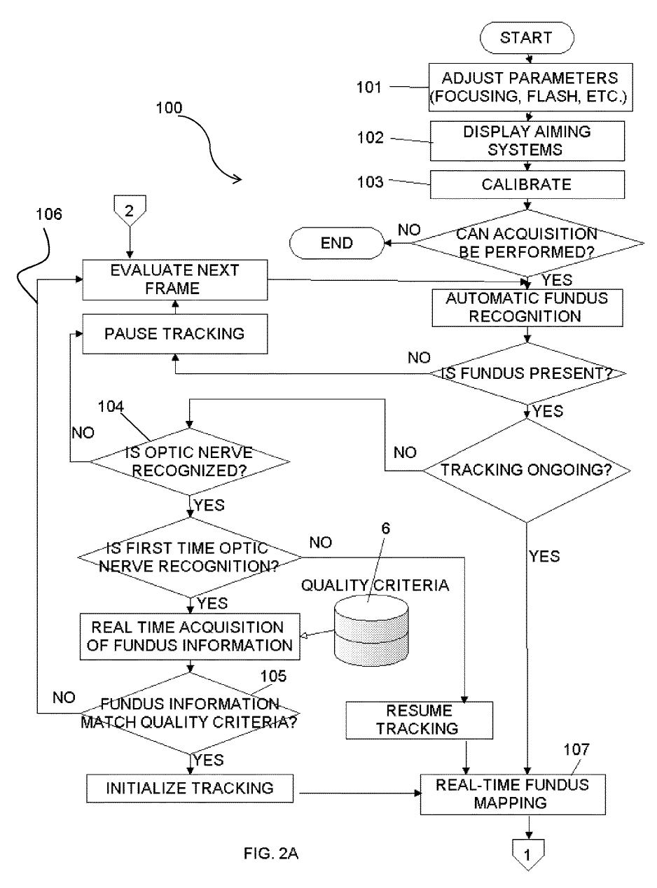

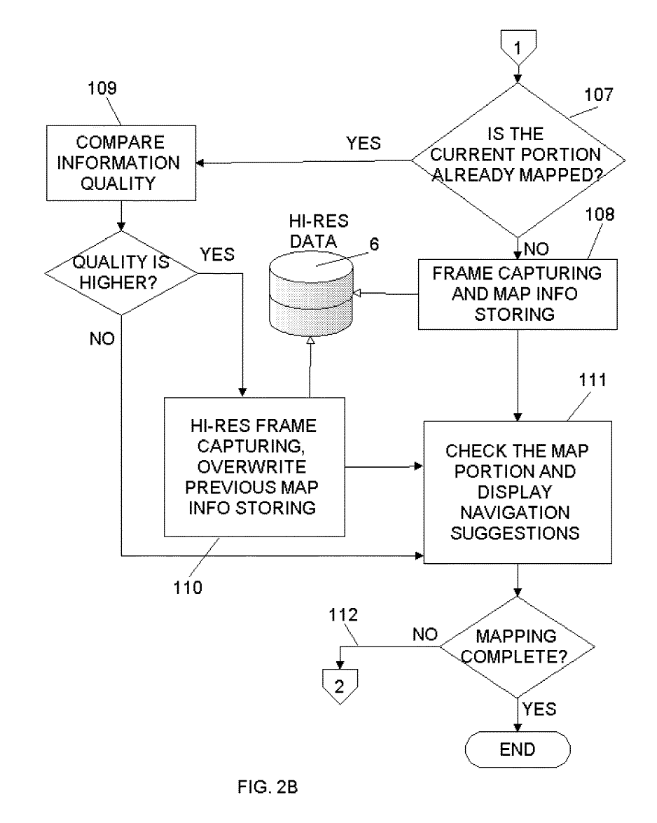

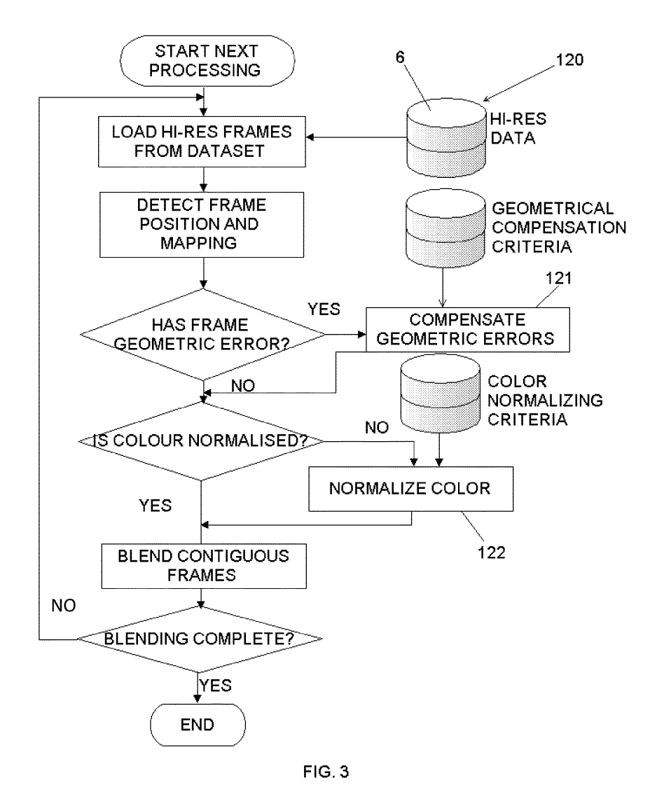

The portable electronic device 1 is loaded with a software application capable of controlling the acquisition and processing of images of an ocular fundus according to a method 100 whose operating procedures are described in detail in the following paragraphs, with reference to the block diagrams of FIGS. 2a, 2b and 3.

The software application is started by the operator after the portable electronic device 1, on which the accessory 5 has already been mounted, has been brought to a predetermined distance from a patient's eye.

On starting, the application, having switched on the video-photocamera 2 and the light source provided to illuminate the ocular fundus of the user via the optical unit of the accessory 5, provides a preliminary step 101 of adjustment of some of the main parameters of the video-photocamera 2, including the luminosity of the video-photocamera 2 and of the light source 7, as well as the focal distance of the images.

Meanwhile, the images received from the video-photocamera 2 are displayed on the display 3 so as to be immediately usable by the operator.

A step 102 is then provided for the display of the eye aiming systems, in which the operator, following the images on the screen, tell the system to be in front of an eye. Following this instruction, the software starts a calibration step 103, in which a process is started for the recognition of some specific physical characteristics of the eye, for example the iris, the pupil, and so on, in order to allow the centring of the pupil and the consequent recording of the images of the ocular fundus. These parameters will subsequently be used in the various steps of the method for recognizing the ocular fundus.

If the preceding steps have yielded a positive outcome, then a step 104 of identifying the optic nerve within the ocular fundus is started.

This step provides for an initial acquisition of successive images, preferably in video form, carried out by the operator inside the pupil, and a simultaneous real-time analysis of the acquired images, in which, image by image, the software attempts to identify the optic nerve (more precisely the head of the optic nerve, or “optic disc”).

The head of the optic nerve is always present in the ocular fundus, and, because of its special circular shape, from which the blood vessels supplying the ocular fundus branch out, and its colouring, which is markedly different from the surrounding fundus, it is a reference point that can be identified with certainty.

When the optic nerve has been identified, the image containing it is analysed in qualitative terms, and, if it conforms to predetermined minimum parameters, is selected and acquired as the initial reference image for the step of mapping the ocular fundus (step 105).

However, if the quality of the image in which the optic nerve has been identified is inadequate, the system proceeds to analyse the next image (step 106).

The quality of the image is preferably evaluated on the basis of the following parameters: the number of details present, colour aberrations, focusing defects, contrast, signal to noise ratio, and contrast to noise ratio.

When the initial reference image has been identified, the actual step of mapping the ocular fundus starts (step 107).

This step provides for the mutual positioning of some images selected from all those acquired by the video-photocamera 2 so as to form a map of the ocular fundus formed by the assembly of the selected images. This is done by assigning to each selected image suitable position coordinates relative to the initial reference image selected previously, in which the presence of the optic nerve was identified, or relative to preceding images that have already been positioned relative to the initial reference image.

The successive images are acquired by the video-photocamera 2 in video form. In a preferred embodiment, the video images are acquired in high resolution, according to the 1080p standard for example, at a frequency of 30 frames per second (fps).

In a preferred version, each image acquired by the video-photocamera 2 is reduced so as to allow a faster analysis of each image.

In an alternative version of the method of the present invention, the acquisition of the images by means of the video-photocamera 2 takes place directly at low resolution.

Each acquired image is analysed and compared with the initial reference image, or with any other images which have already been assigned a position on the map.

Preferably, this comparison is based on the recognition of common portions between the images, on the basis of which the mutual positioning of one image relative to another can be determined.

Portions of image suitable for the recognition process are typically the head of the optic nerve and the various configurations of the blood vessels present on the ocular fundus, as well as other identification characteristics (features) such as gradients, edges and vertices. These identification characteristics can be placed in correspondence between images by means of suitable algorithms.

When the image has been correctly positioned relative to the preceding images, the system checks whether the acquired and positioned image contains portions of ocular fundus which are new relative to the previously selected images. In this case, the image is selected in its turn (step 108).

The selected image is saved in high resolution.

This operation, if the images acquired from the video-photocamera 2 have been acquired in high resolution and then reduced for the mapping analysis, can be carried out by saving the image originally acquired in high resolution and corresponding to the analysed reduced image. Alternatively, if the acquisition of the images by means of the video-photocamera 2 has taken place in low resolution, provision is made to acquire the image to be saved in high resolution at the moment when the low-resolution image is selected.

If the acquired and positioned image shows a portion of the ocular fundus already shown in a previously selected image, a step of qualitative comparison is initiated between the image portions, for the purpose of identifying and selecting the image portion which is qualitatively better.

The portion of the new acquired and positioned image is then compared with the previously selected image in terms of the presence of focusing defects, colour aberrations, contrast, noise, signal to noise ratio, and contrast to noise ratio (step 109). If the quality of the portion of the acquired and positioned image is better than that of the corresponding portion of the previously selected image, it is then selected in its turn and saved in place of the previously selected image (step 110).

This step of comparing the quality of the images is also applicable to the initial reference image, so that a new image containing the representation of the head of the optic nerve can replace the one originally selected and saved.

In a preferred embodiment of the invention, each acquired image is divided into a plurality of areas, such that this step of comparison in terms of quality is executed for each pair of corresponding areas of these images. Thus it is possible for a certain area of an image to be selected and saved in place of a corresponding area of a previously selected image, while the remaining areas are still those of the previously selected image.

At the end of the step of positioning, and possibly selecting, an acquired image, the system checks whether the step of mapping the ocular fundus is or is not complete (step 111).

This analysis is carried out by comparing the set of the images selected up to this point with a predetermined map model within the system. Preferably, this predetermined map model is substantially based on a standardized morphology of the ocular fundus containing known dimensional data such as the diameter of the fundus itself, the diameter of the head of the optic nerve, and the distances between the various main components of the fundus, such as the fovea.

This simplified model, which is generally valid for all patients, may be replaced in another aspect of the invention by a specific personalized model for the patient being examined, reconstructed from previous examinations.

If the system ascertains that the mapping is not yet complete, the next acquired image from the video-photocamera 2 is analysed (step 112), by repeating the analysis process described above.

Additionally, according to another aspect of the present invention, provision is made for the system to simultaneously provide the user with assisted navigation for the purpose of identifying the portions of the ocular fundus whose images have not yet been acquired. The same information can be provided in order to re-acquire images of regions of the ocular fundus for which images of inadequate quality have been acquired previously.

In particular, the system can display on the display 3 the regions of the ocular fundus whose images have already been saved and the regions for which no image is yet present. By way of example, the regions of the ocular fundus whose images have already been acquired can be displayed in a colour and those not yet acquired can be displayed in a different colour.

Thus the operator is given immediate information on the progress of the mapping of the ocular fundus, and can easily deduce where the portable electronic device 1 should be moved in order to acquire the missing regions of the ocular fundus as well, without the trouble of precisely remembering the preceding acquisitions and without spending unnecessary time on regions that have already been acquired.

In a particular embodiment, the system supplies the data for assisted navigation using arrows on the display 3 and audio indications. According to a further aspect of the present invention, the analysis of each acquired image for the purpose of the step of mapping the ocular fundus is carried out within a time interval of not more than 125 milliseconds.

In particular, in this time interval, the system compares the acquired image with the previously selected and positioned images, in order to position it correctly on the map, and also to make a qualitative comparison with the previously selected and positioned images, if necessary.

Preferably, this processing takes place within a time interval of not more than 70 milliseconds.

More preferably, this processing is carried out before the video-photocamera acquires the next image to be analysed.

In the case of a 30 fps video-photocamera, this means that the processing time is not more than about 33 milliseconds.

At the end of the mapping step, the software proceeds to the next step of joining (stitching) the selected and saved images (step 120, FIG. 3) according to the mutual positioning defined by the previous mapping step, so as to generate a single image of the ocular fundus.

The images subjected to the joining step are the high-resolution images saved in the memory 6 during the mapping step.

This joining operation, in turn, comprises various steps of processing the images, since the images saved in high resolution after the mapping step typically show the same problems:
deformation due to the configuration of the eye;
deformation due to the relative positioning of the eye and the portable device;
chromatic difference between different selected images;
difference in luminosity and contrast between the selected images.

In particular, the acquired images of the ocular fundus inevitably show deformation due to the spherical configuration of the fundus.

These deformations may result in macroscopic errors in the final image obtained by joining the various selected images. Consequently a preliminary step 121 of correction of the geometric deformations is provided, in which step each image is analysed and, on the basis of its positioning on the map among other factors, the system can recognize any deformations due to the curvature of the ocular fundus and can compensate for these.

A further step 122 of normalization of the colour and luminosity among the different images is then provided, so that the subsequent step in which the actual joining takes place results in a single image having a uniform colour.

These geometrical and chromatic normalization steps ensure that none of the information about the distinctive diagnostic elements present in the acquired images is lost in any way.

It will be appreciated that this single image has no duplicated or repeated areas, and this significantly reduces the overall volume of data to be saved in the memory 6, and possibly, to be transmitted remotely. When the single image of the ocular fundus has been created, it can easily be transmitted remotely by the portable electronic device 1, using the normal network connections with which it is provided.

This image can then be analysed by a doctor for the appropriate diagnosis.

Thus the present invention resolves the problem of the cited prior art identified above, while also offering numerous other benefits, including immediate and intuitive assistance with the acquisition of the ocular fundus by operators, including non-expert operators, as well as facilitation of the subsequence analysis work carried out by specialist doctors.

Additionally, because of the assistance with navigation offered by the software in the mapping step, the operator can be certain that the final image produced by the joining process contains all the areas of the ocular fundus.

A person skilled in the art can make any possible modifications that may be appropriate to the method described above without departing from the scope of the present invention as defined by the claims below.

1. A method for acquiring and processing images of the ocular fundus by a portable electronic device, the portable electronic device comprising a video-photocamera capable of acquiring a plurality of images at a predetermined frequency, a processor capable of processing the images, and a memory in which the images can be saved, the method comprising:
acquiring a plurality of images of the ocular fundus by the video-photocamera of the electronic device,
mapping certain images selected from the plurality of acquired images, each of the selected images being positioned relative to the other, previously selected, images so as to form a map of the ocular fundus formed by the assembly of the selected images, the positioning of each image relative to the other, previously selected, images being carried out within a maximum time interval of 125 milliseconds, and
joining the selected images or images corresponding to them according to the mutual positioning defined by the mapping, so as to generate a single image of the ocular fundus.

2. The method according to claim 1, wherein the mapping comprises of identifying a head of an optic nerve present in the ocular fundus.

3. The method according to claim 2, wherein identifying the head of the optic nerve comprises analysis of the acquired images in succession until a shape of the head of the optic nerve is identified in one of the acquired images.

4. The method according to claim 3, wherein the quality of the image in which the optic nerve is identified is analysed, and, if it conforms to predefined minimum parameters, the image is selected and acquired as an initial reference image for the aforesaid mapping.

5. The method according to claim 4, wherein, after the definition of the initial reference image, a step of positioning acquired images relative to the initial reference image is carried out, in order to form the map of the ocular fundus.

6. The method according to claim 5, wherein the positioning of acquired images is carried out on the basis of a comparison with the initial reference image or with other, previously positioned, images.

7. The method according to claim 6, wherein the comparison provides for the recognition of common portions of the images.

8. The method according to claim 5, wherein each acquired and positioned image, if it shows a portion of ocular fundus not shown by a previously selected image, is in turn selected to form the map of the ocular fundus.

9. The method according to claim 5, wherein each acquired and positioned image, if it shows a portion of ocular fundus shown by a previously selected image, is:
compared with this previously selected image in terms of image quality, and, if the quality of the acquired and positioned image is better than that of the previously selected image,
selected in place of the previously selected image.

10. The method according to claim 9, wherein each of the acquired images is divided into a plurality of areas and the comparison between images in terms of quality is executed for each pair of corresponding areas of the images.

11. A method according to claim 9, wherein the positioning of each image relative to the other previously selected images, and the comparison which may be made in terms of quality, are executed within a maximum time interval of 70 milliseconds.

12. The method according to claim 11, wherein the positioning of an image relative to the other previously selected images, and the comparison which may be made in terms of quality, are executed before the next image is acquired.

13. The method according to claim 12, wherein the images are acquired by the video camera at a frequency of 30 images per second.

14. The method according to claim 1, wherein the map of the ocular fundus formed by the selected images is compared with a predetermined map model to determine whether the mapping step is complete.

15. The method according to claim 14, wherein provision is made for the predetermined map model to be based on a personalized model for each patient, obtained from examinations carried out previously.

16. A method according to claim 14, wherein provision is made for supplying information to the user if the mapping is incomplete, this information being useful for identifying the portions of the ocular fundus whose images have not yet been acquired.

17. The method according to claim 1, wherein the images are acquired in high resolution and are reduced before being processed during the mapping step.

18. The method according to claim 17, wherein, for each of the selected images, the corresponding high-resolution image is saved.

19. The method according to claim 1, wherein the joining of the selected images or of the corresponding images comprises a step of correcting the geometrical deformations.

20. The method according to claim 1, wherein the joining of the selected images or of the corresponding images comprises a step of colour normalization.

21. A software application, loadable into a portable electronic device comprising a video camera capable of acquiring a plurality of images, a processor capable of processing the images, and a memory in which the images can be saved, the software application being capable of controlling the acquisition and processing of images of the ocular fundus by the portable electronic device according to the method of claim 1.

22. A portable electronic device, comprising a photographic camera or a video camera capable of acquiring a plurality of images, a processor capable of processing the images, and a memory in which the images can be saved, into which is loaded a software application according to claim 21.
